(19) United States
(12) Patent Application Publication
(10) Pub. No.: US 2007/0026479 A1
(43) Pub. Date: Feb. 1, 2007

(54) Liquid protein markers for native gel electrophoresis

(75) Inventors: Thomas Beardslee, Carlsbad, CA (US); Cinnamon Bolz, San Diego, CA (US); Timothy Updyke, Carlsbad, CA (US); Regina Rooney, La Jolla, CA (US)

Correspondence Address: INVITROGEN CORPORATION;C/O INTELLEVATE, P.O. BOX 52050, MINNEAPOLIS, MN, 55402, US

(73) Assignee: Invitrogen Corporation, Carlsbad, CA (US)

(21) Appl. No.: 11/475,260

(22) Filed: Jun. 27, 2006

Related U.S. Application Data
(60) Provisional application No. 60/694,816, filed on Jun. 27, 2005.
(60) Provisional application No. 60/724,668, filed on Oct. 7, 2005.

(51) Int. Cl. C12Q 1/37; C07K 1/26

(52) U.S. Cl. 435/23; 204/450

(57) ABSTRACT

Marker sets are provided for use on nondenaturing gels. The protein molecular weight markers are provided in liquid form, and are stable in liquid form for at least two months at 4 degrees C. and at least one year at −20 degrees C. Methods of using the markers and kits containing stable native protein markers in liquid form for determining molecular mass of proteins using electrophoresis are also provided. Furthermore, methods for generating revenue by selling the liquid molecular weight markers, are provided.

### CROSS REFERENCE TO RELATED APPLICATIONS

This application claims the benefit of priority under 35 U.S.C. § 119(e) of U.S. Provisional Application Ser. No. 60/694,816, filed Jun. 27, 2005 and of U.S. Provisional Application Ser. No. 60/724,668, filed Oct. 7, 2005, the entire contents of which are incorporated herein by reference.

### BACKGROUND OF THE INVENTION

1. Field of the Invention

The invention relates generally to biomolecular separations and more specifically to standards for electrophoretic separations of biomolecules.

2. Background Information

The recent surge in proteomics research activity has brought to light the need for more tools to investigate the properties of newly identified proteins and to serve the fast-growing area of proteomics research studying protein-protein interactions. Among the tools that will assist researchers in this area is native electrophoresis. The ability to maintain native protein conformation and protein complex quaternary structure, coupled with the unparalleled resolution capability of electrophoresis makes this technique a powerful tool for the analysis of protein-protein interactions. Traditional native electrophoresis has enjoyed only limited applicability for native protein analysis because of the high operative pH of the Tris-Glycine system that may adversely affect proteins sensitive to high pH conditions. A technique with more broad applicability and near-neutral operating pH was developed by Schägger and von Jagow (1991) named Blue Native Polyacrylamide Gel Electrophoresis (BN PAGE).

Assessing protein-protein association and conformational status using native gel electrophoresis requires reliable and accurate molecular weight markers. Because the markers must retain a consistent migration profile in the absence of denaturants, they have thus far been unreliable or inconvenient to use, since their stability in liquid form over time is not predictable.

### SUMMARY OF THE INVENTION

The present invention provides sets of protein molecular weight markers for native electrophoresis, in which a molecular weight marker set is provided as a mixture of two or more proteins of different molecular weights in liquid form. In preferred embodiments, the protein molecular weight marker sets of the present invention are stable for at least two months in liquid form at 22 degrees Centigrade. In some preferred embodiments, the protein molecular weight marker sets of the present invention are stable for at least three months in liquid form at 4 degrees Centigrade. In some preferred embodiments, the protein molecular weight marker sets of the present invention are stable for at least one year in frozen liquid form at −20 degrees Centigrade. In some preferred embodiments, the protein molecular weight marker sets of the present invention are stable for at least three years in frozen liquid form at −20 degrees Centigrade.

In some preferred embodiments, the markers include a visually detectable protein marker.

In some preferred embodiments, the markers include a protein that is a protease inhibitor.

In some preferred embodiments, the markers includes a protein that migrates a molecular weight of greater than 700 kDa. In some preferred embodiments, the markers includes a protein that migrates at a molecular weight of greater than 1 megadalton. In some preferred embodiments, the markers includes a protein that migrates at a molecular weight of at least 1.2 megadalton.

In some preferred embodiments, the invention provides sets of protein molecular weight markers for native electrophoresis, in which a molecular weight marker set is provided as a mixture of three or more, four or more, five or more, or six or more proteins of different molecular weights in liquid form. The protein molecular weight marker sets of the present invention are stable for at least one year in frozen liquid form at −20 degrees Centigrade.

In another aspect the invention provides kits that comprise sets of protein molecular weight markers for native electrophoresis, in which a molecular weight marker set is provided as a mixture of two or more, three or more, four or more, five or more, or six or more proteins of different molecular weights in liquid form. The protein molecular weight marker sets of the present invention are stable for at least one year in frozen liquid form at −20 degrees Centigrade. The kits can further include reagents for electrophoresis, such as one or dyes, one or more detergents, one or more electrophoresis buffers, or one or more electrophoresis gels. The kits can further include instructions for use.

In another aspect the invention provides methods for determining the native molecular weight of a protein or protein complex using electrophoresis, in which the migration distance of a protein or protein complex is compared with the migration distance of two or more proteins of a molecular weight marker set of the invention.

In another aspect the invention provides methods of generating revenue by providing a customer with a set of molecular weight standards in liquid form in exchange for consideration, such as money.

### BRIEF DESCRIPTION OF THE DRAWINGS

FIG. 1 shows native markers electrophoresed on different gel systems and stained with Coomassie® G-250. A) Native markers of the invention electrophoresed on (left to right) a 4-16% Blue Native Gel, a 3-12% Blue Native Gel, and a 4-12% Tis-glycine gel. B) Native markers of the invention electrophoresed on 3-8% and 7% acrylamide Tris-Acetate gels. C) Native markers of the invention electrophoresed on 6%, 4-12%, 8-16% and 4-20% acrylamide Tris-Glycine gels.

FIG. 2 shows the same marker formulation diluted to 0.05× of its stock concentration and electrophoresed on 4-12%, 8-16%, and 4-20% acrylamide Tris-Glycine gels and silver stained.

FIG. 3 provides a plot of log MW vs. Rf for proteins from a commercially available HMW marker (diamonds) and liquid native protein standards (squares) electrophoresed on a 4-16% Blue Native gel. The standard curve lines were plotted using a second-order polynomial best fit.

FIG. 4 provides a plot of log MW vs. Rf for proteins from a commercially available HMW marker (diamonds) and liquid native protein standards (squares) electrophoresed on a 4-16% Blue Native gel. The standard curve lines were plotted using a second-order polynomial best fit; equations and R-squared values are shown on the figure.

FIG. 5 provides a plot of log MW vs. Rf for proteins from a commercially available HMW marker (diamonds) and liquid native protein standards (squares) electrophoresed on a 4-12% Tris-Glycine gel. The standard curve lines were plotted using a second-order polynomial best fit; equations and R-squared values are shown on the figure.

FIG. 6 shows different lots of liquid native markers (LNM) of the invention electrophoresed on different gel systems and stained with Coomassie® G-250. Lane 1, commercially available molecular weight markers; Lane 2, LNM lot A, stored at 4 degrees C. for 91 days; Lane 3, LNM lot B, stored at 4 degrees C. for 67 days; Lane 4, LNM lot B, stored at 22 degrees C. for 67 days; Lane 5, LNM lot B, stored at 30 degrees C. for 67 days; Lane 6, LNM lot C, stored at 4 degrees C. for 66 days; Lane 7, LNM lot C, stored at 22 degrees C. for 66 days; and Lane 8, LNM lot C, stored at 30 degrees C. for 66 days. A) Native markers of the invention electrophoresed on 3-12% Blue Native Gels. B) Native markers of the invention electrophoresed on 4-16% Blue Native gels. C) Native markers of the invention electrophoresed on 4-12% Tris-Glycine gels.

### DETAILED DESCRIPTION OF THE INVENTION

Definitions

In the description that follows, a number of terms used in recombinant DNA technology and protein chemistry are utilized extensively. In order to provide a clear and consistent understanding of the specification and claims, including the scope to be given such terms, the following definitions are provided.

As used herein, the articles “a,” “an” and “one” mean “at least one” or “one or more” of the object to which they refer, unless otherwise specified or made clear by the context in which they appear herein.

As used herein, the terms “about” or “approximately” when referring to any numerical value are intended to mean a value of ±10% of the stated value. For example, “about 50° C.” (or “approximately 50° C.”) encompasses a range of temperatures from 45° C. to 55° C., inclusive. Similarly, “about 100 mM” (or “approximately 100 mM”) encompasses a range of concentrations from 90 mM to 110 mM, inclusive.

As used herein “native” means nondenaturing or nondenatured, and refers to 1) conditions that do not disrupt intermolecular interactions within peptides or proteins that allow them to maintain a three dimensional structure that is either a three dimensional structure of the protein as found in nature or synthesized in a cell-free in vitro translation system, or 2) to proteins having a three dimensional structure that is the same or substantially the same as a three dimensional structure of the protein as found in nature or synthesized in a cell-free in vitro translation system. A three dimensional structure can be a secondary, tertiary, or quaternary structure of a protein.

A “native gel” or “nondenaturing gel” is a gel that does not include denaturing agents such as denaturing detergents (for example, anionic detergents such as SDS or LDS) or chaotropes (urea, formamide, guanidine, potassium iodide, etc) that disrupt protein structure.

As used herein, “native molecular weight markers”, “native markers”, and “native protein standards” refer to two or more proteins that are in nondenatured form, in which the two or more proteins have different molecular weights and can be separated by at least one protein separation process, such as but not limited to, native gel electrophoresis.

The term “label” as used herein refers to a chemical moiety or protein that is directly or indirectly detectable (e.g. due to its spectral properties, conformation or activity) when attached to a target or compound and used in the present methods. The label can be directly detectable (fluorophore, chromophore) or indirectly detectable (hapten or enzyme). Such labels include, but are not limited to, radiolabels that can be measured with radiation-counting devices; pigments, dyes or other chromophores that can be visually observed or measured with a spectrophotometer; spin labels that can be measured with a spin label analyzer; and fluorescent labels (fluorophores), where the output signal is generated by the excitation of a suitable molecular adduct and that can be visualized by excitation with light that is absorbed by the dye or can be measured with standard fluorometers or imaging systems, for example. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a calorimetric substrate (e.g., tetramethylbenzidine (TMB)) or a fluorogenic substrate such as Amplex Red reagent (Molecular Probes, Inc.) to detect the presence of HRP. Numerous labels are know by those of skill in the art and include, but are not limited to, particles, dyes, fluorophores, haptens, enzymes and their calorimetric, fluorogenic and chemiluminescent substrates and other labels that are described in RICHARD P. HAUGLAND, MOLECULAR PROBES HANDBOOK OF FLUORESCENT PROBES AND RESEARCH PRODUCTS (9^{th} edition, CD-ROM, September 2002), supra.

The term “directly detectable” as used herein refers to the presence of a material or the signal generated from the material is immediately detectable by observation, instrumentation, or film without requiring chemical modifications or additional substances.

A “dye” is a visually detectable label. A dye can be, for example, a chromophore or a fluorophore. A fluorophore can be excited by visible light or non-visible light (for example, UV light).

“Amino acid” refers to the twenty naturally-occurring amino acids, as well as to derivatives of these amino acids that occur in nature or are produced outside of living organisms by chemical or enzymatic derivatization or synthesis (for example, hydoxyproline, selenomethionine, azido amino acids, etc.

“Conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having acidic side chains is glutamic acid and aspartic acid; a group of amino acids having amino-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. Preferred conservative amino acid substitution groups are: valine-leucine-isoleucine; phenylalanine-tyrosine; lysine-arginine; alanine-valine; glutamic acid-aspartic acid; and asparagine-glutamine.

As used herein, “protein” means a polypeptide, or a sequence of two or more amino acids, which can be naturally-occurring or synthetic (modified amino acids, or amino acids not known in nature) linked by peptide bonds. “Peptide” specifically refers to polypeptides of less than 10 kDa. As used herein, the term “protein” encompasses peptides. In the context of the present invention, the term “protein” can refer to a multisubunit protein complex.

“Naturally-occurring” refers to the fact that an object having the same composition can be found in nature. For example, a polypeptide or polynucleotide sequence that is present in an organism, including viruses, that can be isolated from a source in nature, and that has not been intentionally modified in the laboratory is naturally-occurring.

A nucleic acid (or nucleotide) or protein (or amino acid) sequence that is “derived from” another nucleic acid (or nucleotide) or protein (or amino acid) sequence is either the same as at least a portion of the sequence it is derived from, or highly homologous to at least a portion of the sequence it is derived from. An amino acid sequence derived from the sequence of a naturally-occurring protein can be referred to as a “naturally-occurring protein-derived amino acid sequence”. A nucleic acid sequence derived from the sequence of a naturally-occurring nucleic acid can be referred to as a “naturally-occurring nucleic acid-derived nucleic acid sequence”. “Highly homologous” in this context means that the sequence is at least 80% identical at the amino acid level, preferably 90% identical at the amino acid level, and more preferably is at least 95% identical at the amino acid level. In the context of protein standards of the invention, two nucleic acid sequences are “homologous” when they are at least 65% identical, preferably at least 70% identical, and are highly homologous when they are at least 80% identical, and more preferably at least 90% identical.

“Recombinant methods” are methods that include the manufacture of or use of recombinant nucleic acids (nucleic acids that have been recombined to generate nucleic acid molecules that are structurally different from the analogous nucleic acid molecule(s) found in nature). Recombinant methods can employ, for example, restriction enzymes, exonucleases, endonucleases, polymerases, ligases, recombination enzymes, methylases, kinases, phosphatases, topoisomerases, etc. to generate chimeric nucleic acid molecules, generate nucleotide sequence changes, or add or delete nucleic acids to a nucleic acid sequence. Recombinant methods include methods that combine a nucleic acid molecule directly or indirectly isolated from an organism with one or more nucleic acid sequences from another source. The sequences from another source can be any nucleic acid sequences, for example, gene expression control sequences (for example, promoter sequences, transcriptional enhancer sequences, sequence that bind inducers or promoters of transcription, transcription termination sequences, translational regulation sequences, internal ribosome entry sites (IRES's), splice sites, poly A addition sequences, poly A sequences, etc.), a vector, protein-encoding sequences, etc. The nucleic acid sequences from a source other than the source of the nucleic acid molecule directly or indirectly isolated from an organism can be nucleic acid sequences from or within the genome of a different organism. Nucleic acid sequences in the genome can be chromosomal or extra-chromosomal (for example, the nucleic acid sequences can be episomal or of an organelle genome). Recombinant methods also includes methods of introducing nucleic acids into cells, including transformation, viral transfection, etc. to establish recombinant nucleic acid molecules in cells. “Recombinant methods” also includes the synthesis and isolation of products of nucleic acid constructs, such as recombinant RNA molecules and recombinant proteins. “Recombinant methods” is used interchangeably with “genetic engineering” and “recombinant [DNA] technology”.

A “recombinant protein” is a protein made from a recombinant nucleic acid molecule or construct. A recombinant protein can be made in cells harboring a recombinant nucleic acid construct, which can be cells of an organism or cultured prokaryotic or eukaryotic cells, or can made in vitro using, for example, in vitro transcription and/or translation systems.

“Do not differ substantially” or “substantially the same” means that the referenced compositions or components differ by less than 10% of the larger of the compared values.

The term “purified” as used herein refers to a preparation of a protein that is essentially free from contaminating proteins that normally would be present in association with the protein, e.g., in a cellular mixture or milieu in which the protein or complex is found endogenously such as serum proteins or cellular lysate.

“Substantially purified” refers to the state of a species or activity that is the predominant species or activity present (for example on a molar basis it is more abundant than any other individual species or activities in the composition) and preferably a substantially purified fraction is a composition wherein the object species or activity comprises at least about 50 percent (on a molar, weight or activity basis) of all macromolecules or activities present. Generally, a substantially pure composition will comprise more than about 80 percent of all macromolecular species or activities present in a composition, more preferably more than about 85%, 90%, or 95%.

The term “sample” as used herein refers to any material that may contain a biomolecule or an analyte for detection or quantification.

An “antimicrobial agent” is a compound that destroys or inhibits the growth of one or more microorganisms, which can be prokaryotic (for example, bacteria) or eukaryotic (for example, fungi). An antimicrobial agent can be a salt or metal, such as, for example, silver or mercury, an azide compound such as sodium azide, or a naturally-occurring or synthetic compound, such as an organic antibiotic.

A “customer” refers to any individual, institution, or business entity, such as a corporation, university, or organization, including a government entity or organization seeking to obtain genomic and proteomic products and services. A customer typically provides consideration, typically by paying money to a provider for a product or a service.

A “provider” refers to any individual, institution, business entity such as a corporation, university, or organization, including a government entity or organization, seeking to provide genomic and proteomic products and services. A provider typically receives consideration, typically monetary consideration, for providing a product or service to a customer. A provider typically provides a product or service in commerce to be sold and, with respect to products, shipped, either directly or indirectly to a customer.

A “commercial product” is a product that is sold and/or shipped through a stream of commerce. For example, a commercial product is typically sold and shipped, either directly, or indirectly using a third party, by a provider to a customer.

Liquid Native Protein Marker Sets

_\ The present invention provides sets of protein molecular weight markers for native gel electrophoresis, in which a native protein molecular weight marker set is provided as a mixture of two or more proteins of different molecular weights in liquid form. By “liquid form” is meant that the proteins of the set are in solution, although the protein molecular weight marker set may be provided as a frozen solution. The two or more proteins of the mixture that have different molecular weights are selected such that their migration on nondenaturing (“native”) gels is relative to their molecular weights. Thus, for the entire set of proteins of the native molecular weight markers, the proteins of the set migrate in the reverse order of their molecular weights, that is, with a smaller molecular weight protein of the proteins of the set migrating a greater distance in a given period of time than a larger molecular weight protein. Preferably, migration of the at least two proteins of a liquid native protein marker set on nondenaturing gels is a function of their molecular weights, that is, the migration of the proteins of a liquid native protein marker set in native gel electrophoresis can be plotted versus molecular weight (or a function of molecular weight, such as for example the log of molecular weight) and a curve and a formula for the curve of the migration versus molecular weight can be generated that has an R² value of greater than 0.9, preferably greater than 0.95 more preferably greater than 0.97, 0.98, or 0.99.

A liquid native protein marker set preferably comprises at least two proteins, and preferably at least three proteins, of different molecular weights that migrate as a function of their molecular weights. One or more of the proteins can be a multisubunit protein. A multisubunit protein can be present in a liquid native protein marker set in more than one form, where “more than one form” refers to different subunit composition. One or more of the proteins can be a multisubunit protein that is present in more than one form, in which the different forms of the multisubunit protein have different molecular weights, and the different forms of the multisubunit protein migrate as a function of their molecular weights. A liquid native protein marker can comprise two, three, four, five, six, seven, eight, nine, ten, eleven, twelve, thirteen, fourteen, fifteen, sixteen, seventeen, eighteen, nineteen, twenty, or more than twenty proteins of different molecular weights that migrate as a function of their molecular weights, one or more of which can be a multisubunit protein. A multisubunit protein can be present in a liquid native protein marker set in more than one form. Different forms of a multisubunit liquid native protein marker set can have different molecular weights, in which the forms of a multisubunit protein migrate as a function of their molecular weights.

In some embodiments, a liquid native protein marker set comprises two or more proteins having molecular weights between 5 kilodaltons (kDa) and 2000 kDa, or between 8 kDa and 1500 kDa. Each of the proteins typically has a different molecular weight that can be distinguished on a native polyacrylamide gel.

In some preferred embodiments, a liquid native protein marker set comprises at least two proteins having molecular weights ranging from 50 kDa or less to 250 kDa or more. In some preferred embodiments, a liquid native protein marker set comprises at least three proteins having molecular weights ranging from 50 kDa or less to 250 kDa or more. In some preferred embodiments, a liquid native protein marker set comprises at least three proteins having molecular weights ranging from 50 kDa or less to 500 kDa or more. In some preferred embodiments, a liquid native protein marker set comprises at least three proteins having molecular weights ranging from 20 kDa or less to 250 kDa or more. In some preferred embodiments, a liquid native protein marker set comprises at least three proteins having molecular weights ranging from 20 kDa or less to 500 kDa or more. In some preferred embodiments, a liquid native protein marker set comprises at least three proteins having molecular weights ranging from 20 kDa or less to 700 kDa or more. In some preferred embodiments, a liquid native protein marker set comprises at least three proteins having molecular weights ranging from 50 kDa or less to 700 kDa or more. In some preferred embodiments, a liquid native protein marker set comprises at least three proteins having molecular weights ranging from 20 kDa or less to 1,000 kDa or more. In some preferred embodiments, a liquid native protein marker set comprises at least three proteins having molecular weights ranging from 50 kDa or less to 700 kDa or more.

The marker set can include, for example, between two and fifteen proteins, and in certain illustrative examples includes between six and ten proteins, between 5 kDa and 2000 kDa, or between 8 and 1500 kDa, or between 20 kDa and 1500 kDa. Each of the proteins typically has a different molecular weight that can be distinguished on a native polyacrylamide gel.

In some preferred embodiments, a liquid native protein marker set comprises at least three proteins having molecular weights ranging from 20 kDa or less to 250 kDa or more, in which the migration of the proteins of a liquid native protein marker set in native gel electrophoresis can be plotted versus molecular weight (or a function of molecular weight, for example, the log of molecular weight) and a curve of the migration/molecular weight can be generated that has an R² value of greater than 0.9, preferably greater than 0.95 more preferably greater than 0.97, 0.98, or 0.99. The marker set can include, for example, between two and fifteen proteins, and in certain illustrative examples includes between six and ten proteins, having molecular weights ranging from 20 kDa or less to 500 kDa or more, in which the migration of the proteins of a liquid native protein marker set in native gel electrophoresis can be plotted versus molecular weight and a curve of the migration/molecular weight can be generated that has an R² value of greater than 0.9, preferably greater than 0.95 more preferably greater than 0.97, 0.98, or 0.99. In some preferred embodiments, a liquid native protein marker set comprises at least six proteins having molecular weights ranging from 20 kDa or less to 750 kDa or more, in which the migration of the proteins of a liquid native protein marker set in native gel electrophoresis can be plotted versus molecular weight and a curve of the migration/molecular weight can be generated that has an R² value of greater than 0.9, preferably greater than 0.95 more preferably greater than 0.97, 0.98, or 0.99. In some preferred embodiments, a liquid native protein marker set comprises at least six proteins having molecular weights ranging from 20 kDa or less to 1,200 kDa or more, in which the migration of the proteins of a liquid native protein marker set in native gel electrophoresis can be plotted versus molecular weight and a curve of the migration/molecular weight can be generated that has an R² value of greater than 0.9, preferably greater than 0.95 more preferably greater than 0.97, 0.98, or 0.99.

As used herein, a “protein” can be a protein or a peptide, and can have multiple subunits that are covalently or noncovalently bound to one another. In the context of the present invention, the term protein also includes protein complexes. Under nondenaturing conditions, the subunits of a multisubunit protein or protein complex preferably remain bound. The native molecular weight of a protein or protein complex is the molecular weight of the protein or protein complex in which the subunits or component proteins are not dissociated. However, it is within the scope of the present invention to have a protein in more than one form, for example, having two or more forms characterized by different subunit compositions and different molecular weights. For example, a protein may be present in a marker set as a dimer and tetramer, or a pentamer and a hexamer, etc.

Proteins of a liquid native protein marker set of the present invention can be naturally-occurring proteins of prokaryotic or eukaryotic origin that can be isolated from natural sources, such as organisms, tissue (including blood, plasma, or serum), cultured cells, or the media of cultured cells. Proteins of a liquid native protein marker set of the present invention can be recombinant proteins synthesized in cultured cells which can be prokaryotic or eukaryotic cells, and can be the same or different from the cells the protein is naturally expressed in, or can be synthesized in in vitro translation systems. A protein of a liquid native protein marker set can be a variant of a naturally-occurring protein, having, for example, at least 70%, at least 80%, at least 90%, at least 95%, or at least 99% sequence identity at the amino acid level with a naturally-occurring protein. A protein of a liquid native protein marker set can be an engineered protein, that may or may not have sequence identity with one or more naturally-occurring proteins.

The proteins of a liquid native protein marker set of the present invention migrate in reverse order of their molecular weights under nondenaturing electrophoresis conditions, such that by using a protein molecular weight marker set of the present invention on the same gel as a sample that includes one or more proteins or protein complexes, a researcher can estimate the native molecular weight of the one or more sample proteins or protein complexes by comparing migration distances of the proteins of the molecular weight marker set with the migration distances of the one or more sample proteins or protein complexes in nondenaturing electrophoresis.

In preferred embodiments, the proteins of the set are pre-mixed in a liquid formulation such that the proteins do not require solubilization in buffer by the user prior to use, nor do individual protein solutions have to be mixed together to obtain the set having proteins of different molecular weights in the appropriate proportions. The proteins of the molecular weight marker set proteins in some preferred embodiments are present in a concentration such that an aliquot of the liquid protein molecular weight marker set can be loaded directly on a native gel. Each protein of the set is present at a concentration that can be visualized either directly or when the protein is stained (for example, with protein stains or dyes such as but not limited to a Coomassie®, SYPRO®, or silver stain). For example, the concentration of a given component protein of a native protein molecular weight marker set can be from about 5 microgram per milliliter (mL) to about 10 milligrams per mL, or preferably from about 10 micrograms per mL to about 5 mgs per mL, more preferably from about 50 micrograms per mL to about 2 mgs per mL, and more preferably yet from about 100 micrograms per mL to about 1 mg per mL. Different component proteins of a native protein marker set can be present at different concentrations.

In preferred embodiments, a liquid native protein marker set includes proteins in a buffer compatible with native electrophoresis (for example, Tris-glycine native gels, Tris-actate native gels, or Blue Native gels), in which a nondenaturing “heavy” compound (such as glycerol) and, optionally, a dye can be present for convenient loading into a sample well. Other compounds, such as but not limited to salts, antioxidants, antimicrobial agents, reducing agents, chelating agents, or protease inhibitors can also optionally be present. The liquid marker protein solution can also include one or more nondenaturing detergents. Preferably, the buffer in which the proteins are dissolved is at or near neutrality, that is at a pH greater than 6 and less than 8, and more preferably at a pH between about 6.5 and 7.5. An exemplary solution for a liquid native marker set is 50 mM BisTris/HCl pH 7.0, 50 mM NaCl, 10% w/v Glycerol, 0.001% Ponceau S.

The stability of the proteins of the liquid native marker set allows for this “ready to use” convenience. As used herein, “stability” means that the protein is not degraded (for example, proteolyzed) such that its molecular weight changes, or denatured such that the migration of the protein in a native gel changes. Denaturation of a protein can cause gel migration changes due to, for example, dissociation of subunits, altered protein conformation, aggregation, or any combination of these.

A liquid native marker set of the present invention is stable for at least one year at −20 degrees C. or lower, such as for at least three years at -20 degrees C. or lower. Preferably, a liquid native marker set is stable for at least one month at a temperature of 4 degrees C. or lower. Preferably, a liquid native marker set is stable for at least two months at a temperature of 4 degrees C. or lower. In some aspects a liquid native marker set is stable for at least 2, 3, 4, 5, 6, 7, 8, 9, 10, 11, or 12 months at 4 degrees Centigrade or −20 degrees Centigrade up to between 2, 3, 4, 5, 6, 7, 8, 9, 10, 11, 12, 13, 14, 15, 16, 17, 18, 24, 36, 48, and 60 months or longer at 4 degrees Centigrade or −20 degrees Centigrade.

These criteria for stability can be based on testing of lots of the marker sets using elevated temperature and accelerated aging tests. Calculation of equivalent storage time for accelerated aging is performed assuming a Q₁₀ of 2.0 and the following equation:
Equivalent storage time in days at T_{L}=2.0^{[(TH-TL)/10]}×Time in days stored at T_{H}
Where T_{L} is the lower temperature in Celsius and T_{H} is the higher temperature in Celsius. For instance, 30 days storage at 30° C. is equivalent to 960 days at −20° C. (2.0^{[(30--20)/10]}×30=960).
Migration Consistency

A set of liquid native molecular weight markers is stable such that, after storage of an aliquot of a lot of a marker set for a given period of time at a given temperature, when the marker set is electrophoresed on a gel and another lot of a marker set previously tested to be stable is run in an adjacent lane, the Rf (migration) for a marker band of the test lot matches that of the same marker band for a lot previously judged to be stable, within a tolerance of +/−0.02 Rf (Retardation factor) units, where an Rf unit is the distance migrated by an individual protein divided by the total possible migration distance.

Minimal Non-Marker Bands

Preferably, a lot of a set of liquid native molecular weight markers is stable such that, after storage for a given period of time at a given temperature, when the marker set is electrophoresed on a gel, and the gel is stained, and scanning and image analysis (densitometry) are performed, the intensity (peak height) for any non-marker band is not greater than 20% of the intensity of the least intense (smallest peak height) of the two nearest marker bands.

Consistent Electrophoresis Pattern

Preferably, a lot of a set of liquid native molecular weight markers is stable such that, after storage for a given period of time at a given temperature, when the marker set is electrophoresed on a gel, and the gel is stained, visual inspection of the marker set does not reveal any other differences in performance (such as but not limited to band sharpness) when compared with the performance of a previous lot of liquid native markers judged to be stable by these criteria.

The marker sets of the invention are stable, in that lots of the marker sets are able to pass these criteria of consistent migration, minimal non-marker bands, and consistent electrophoresis pattern in accelerated aging tests, such that the marker sets are reliably stable in liquid (frozen) form at −20 degrees C. for at least six months.

In preferred embodiments, the protein molecular weight marker sets of the present invention are stable for at least one year in liquid (frozen) form at −20 degrees Centigrade. In some preferred embodiments, the protein molecular weight marker sets of the present invention are stable for at least three years in liquid (frozen) form at −20 degrees Centigrade. In some preferred embodiments, the liquid protein molecular weight marker sets of the present invention are stable for at least one month in liquid form at 4 degrees Centigrade. In some preferred embodiments, the liquid protein molecular weight marker sets of the present invention are stable for at least two months in liquid form at 4 degrees Centigrade. In some preferred embodiments, the liquid protein molecular weight marker sets of the present invention are stable for at least three months in liquid form at 4 degrees Centigrade. In some preferred embodiments, the liquid protein molecular weight marker sets of the present invention are stable for at least six months in liquid form at 4 degrees Centigrade.

Preferably, a liquid native protein molecular weight marker set as provided herein has a shelf-life of at least 1 year at −20 degrees Centigrade. In some preferred embodiments, a liquid native protein molecular weight marker set as provided herein has a shelf-life of at least 1 month at 4 degrees Centigrade. Shelf life means that the commercial product performs in a way that meets the expectations of a majority of customers. In the present case, the liquid native markers perform consistently enough over time so as to be acceptable to a customer, having no more than a 5% change in migration over the shelf life. Preferably, the markers have no more than a 2.5% change in migration over the shelf life, more preferably, no more than a 2% change in migration over the self life, more preferably yet no more than a 1% change in migration over the shelf life, and even more preferably no more than a 0.2% change in migration over the shelf life.

Preferably, the liquid native markers maintain band intensity and pattern over time so as to be acceptable to a customer, having no more than a 20% change in the width or intensity of a given band over the shelf life. In some preferred embodiments, the markers have no more than a 10% change in band intensity and pattern over the shelf life.

The proteins of a liquid native molecular weight marker set of the present invention, in addition to being stable in native conformation in liquid form, are selected for their property of providing good migration properties and band resolution in Tris-glycine and Blue Native gel electrophoresis systems. For Tris-glycine gels in particular, this requires that the proteins have a pI lower than the operating pH of the electrophoresis conditions (Tris-glycine native electrophoresis conditions produce an operating pH of the gel system of approximately 9.3), such that their migration to the anode is not impaired by their charge in the buffer system. Sharp band resolution is advantageous when calculating migration distances and identifying bands.

In preferred embodiments, a protein molecular marker set for native gel electrophoresis includes three or more proteins, four or more proteins, five or more proteins, or six or more proteins. The inclusion of multiple proteins in a marker formulation increases the range and accuracy of molecular weight determination of sample proteins. The combination of as many as six proteins, each of which exhibits one or more highly resolved bands in native electrophoresis systems, in a single native marker formulation in liquid ready-to-load form, such that each protein of the mixture is stable for at least one month at 4 degrees C., provides increased accuracy of protein molecular weight determination at greater convenience to the user.

A set of native liquid molecular weight markers of the present invention is formulated of a set of proteins that are stable in liquid form and cover a desirable molecular weight range. In preferred embodiments, a liquid native protein marker set includes three or more proteins of different molecular weights. In some preferred embodiments, a liquid native protein marker set includes four or more, five or more, or six or more proteins of different molecular weights. One or more of the proteins of the marker set can be present in more than one oligomeric form. The proteins can span a molecular weight range of up to 50 kDa, up to 100 kDa, up to 200 kDa, up to 400 kDa, or up to 800 kDa. In a preferred embodiment, the proteins of a liquid native protein marker set span a molecular weight range of 1 megadalton or greater.

Oligomeric proteins used in a native liquid molecular weight marker set can have any number of subunits. The subunits of an oligomeric protein can be the same or different. In some cases, one or more proteins of the molecular weight marker set is provided in more than one native form, where the different native forms have different molecular weights. For example, multisubunit proteins can be present in the marker set in native forms having different subunit composition or different oligomeric forms, such that they produce more than one marker band on a gel.

The present invention provides a set of protein molecular weight markers in which one or more of the proteins of the set has a native molecular weight greater than or equal to 700 kildaltons. The present invention also provides protein molecular weight marker sets in which one or more of the proteins of the set has a native molecular weight greater than or equal to 1 megadalton (1000 kDa). The present invention also provides protein molecular weight marker sets in which one or more of the proteins of the set has a native molecular weight greater than or equal to 1.2 megadalton (1200 kDa).

A component protein of a liquid native molecular weight marker set can be provided at a concentration of from about 0.05 to about 5 mg/ml, and preferably at a concentration of from about 0.1 to about 2.5 mg/ml, and more preferably at a concentration of about at a concentration of from about 0.2 to 1 mg/ml. The concentration of each protein component of the liquid marker set is preferably optimized to obtain electrophoresis bands that are of adequate resolution and intensity when visualized with a protein stain.

As another novel aspect, the present invention provides sets of molecular weight markers in which at least one of the proteins of the set is visually detectable. For example, one or more of the proteins of a molecular weight marker set for native protein gels can include one or more chromophores or fluorophores. A protein of a liquid native marker set can be naturally fluorescent protein, such as, for example, green fluorescent protein (GFP) or a variant thereof, including variants having increased brightness, or altered absorption and emission wavelengths with respect to wild-type GFP, or red fluorescent protein (Ds red or other anthozoan fluorescent proteins) or a variant thereof, including variants having increased brightness, or altered absorption and emission wavelengths with respect to wild-type fluorescent anthozoan proteins. In some aspects of the present invention, a phycobilisome protein is one of the protein molecular weight markers. For example, phycocyanin, allophycocyanin, or phycoerythrin can be one of the proteins of a molecular weight marker set. Phycoerythrin, such as, for example, phycoerythrin R, B, or Y, has the advantage of a readily visualizable red color that can allow a user to immediately recognize its position on a gel. This allows the user to identify the proteins at each location in the marker lane, since they migrate in order of their native molecular weights. The protein can be used for assessing the migration rate or position of stained or unstained proteins of the sample even while the gel is running. The present invention provides a liquid native marker set that includes multiple proteins that are stable in liquid form for at least two months at 4 degrees C., and at least one year at −20 degrees C., in which at least one of the proteins is visually detectable in its native, unstained form on a gel.

In some preferred embodiments, the liquid native protein molecular weight marker set of the present invention includes at least one protease inhibitor as one of the protein markers. The protease inhibitor can contribute to the stability of the marker set by inhibiting any contaminating protease, for which it is specific, that may be present. For example, trace amounts of a protease may be present in the preparation of one or more proteins of the marker set. The protease inhibitor can be an inhibitor of cysteine proteases, serine proteases, aspartic proteases, or metalloproteases. For example, the protein marker set can include a trypsin inhibitor as a protein molecular weight marker, such as, for example, egg white trypsin inhibitor or soybean trypsin inhibitor.

Methods of Use

During native gel electrophoresis, the proteins of a set of molecular weight markers of the present invention migrate at a rate that is a function of their native molecular weights. Thus the native protein liquid markers of the present invention can be used to estimate the native molecular weight of a protein or protein complex electrophoresed in the same electrophoresis run using the distances the protein markers and proteins of the sample migrated. For example, the distance a sample protein migrated during electrophoresis can be compared with the distance one or more marker proteins migrated during electrophoresis to determine a relative size for the sample protein. In another example, the molecular weight (or a function of molecular weight) of proteins of the set can be plotted as a direct or indirect function of their migration distance obtained from running the liquid protein marker set on a native gel. For example, the log of the molecular weights of proteins of the set can be plotted as a function of their migration distance obtained from running the liquid protein marker set on a native gel. The curve generated can be used to determine the estimated molecular weight of a sample protein using its migration distance in the same gel(s). In one instance, the log of the molecular weight of each protein of a set of protein molecular weight markers can be plotted as a function of the migration distance, and the native molecular weight of one or more sample proteins or protein complexes can be estimated using the generated native molecular weight marker curve and the migration distances of one or more sample proteins or protein complexes run on the same gel.

The determination of molecular weights on a native gel system is not exact, but can provide a useful estimate of the molecular weight of one or more proteins of interest in native form. The liquid native marker sets of the present invention can be used for determining the estimated or relative molecular weight of a protein or protein complex under nondenaturing conditions, where the protein of interest and molecular weight marker set are loaded separately on a nondenaturing gel. The protein sample and liquid marker set are electrophoresed on the gel, and the migration distances of the marker proteins and one or more sample proteins are measured. The molecular weights, or the log of the molecular weights, of the proteins are plotted as a function of their migration distances. The molecular weight of a sample protein is determined by locating the value on the curve that corresponds to its migration distance.

In some preferred embodiments, a protein or protein complex of interest or a sample of interest can be run on multiple native gels of different acrylamide concentrations. In each electrophoresis run, a set of marker proteins of the present invention is run alongside the sample on the same gel. The migration of each marker at the different acrylamide concentrations is used to estimate molecular weights of one or more sample proteins or complexes using Ferguson plots, as is known in the art (Gallagher “Native Discontinuous Electrophoresis and Generation of Molecular Weight Standard Curves (Ferguson Plots)” in Current Protocols in Protein Science, Coligan et al., eds. John Wiley and Sons, N.Y. (1995); and Andrews, Electrophoresis: Theory, Techniques and Biochemical and Clinical Applications, 2^{nd} ed. Oxford University Press, N.Y, (1986).

Native gels can be made of any polymer or combination of polymers that produces a gel in which the pore size is relatively consistent (or within a consistent range) for a given polymer concentration. Polyacrylamide gels are exemplary gels for the separation of proteins. The polyacrylamide gels can be of any concentration that allows for separation of proteins of interest. For example, polyacrylamide gels can range from about 1.5% to about 30% polyacrylamide, preferably range from about 2% to about 25% polyacrylamide, and in even more preferred embodiments of the present invention, range from about 2.5% to about 20% polyacrylamide. Polyacrylamide gels typically also comprise a lesser percentage of a cross-linking polymer, such as, for example, N,N′-methylenebisacrylamide. In some preferred embodiments, polyacrylamide gels are gradient gels in which the concentration of polyacrylamide in the gel varies as a concentration gradient from a low to a high concentration. For example, a gradient gel used in native protein electrophoresis can have a concentration gradient that ranges from 3% to 12% polyacrylamide, or from 4% to 16% polyacrylamide. Methods of making and using polymeric gels for protein and peptide separation, including making and using nondenaturing gels for protein and peptide separation, are well known in the art.

For example, Tris-glycine or Tris-acetate gels can be used for native protein separation, or “blue native” (BN) gels that use Bis-tris polyacrylamide gels can be used. Tris-glycine and Tris-acetate gels for native protein separation are known in the art. The use of blue native gels, in which the cathode buffer, the protein sample buffer, or both, contain Coomassie® G-250 dye, is also known in the art and described in Schagger H and von Jagow G (1991) “Blue native electrophoresis for isolation of membrane protein complexes in enzymatically active form” Anal. Biochem. 199: 223-231; Schagger H, Cramer W A, and von Jagow G (1994) “Analysis of molecular masses and oligomeric states of protein complexes by blue native electrophoresis and isolation of membrane protein complexes by two-dimensional native electrophoresis” Anal. Biochem. 217: 220-230; and Schagger H (2001) “Blue-native gels to isolate protein complexes from mitochondria” Methods Cell Biol. 65: 231-244. In this electrophoresis method, Coomassie® G-250 dye binds proteins in their native state, conferring a negative charge to the proteins. The negatively charged Coomassie®-stained proteins migrate to the anode at a velocity that is proportional to their molecular weight.

Native protein markers can also be used as standards in other protein separation and analysis techniques, such as but not limited to: isolelectric focusing in semi-solid (e.g. gel) or liquid media; chromatography, including chromatographic separation based on size, charge, or a combination thereof, including HPLC and FPLC; and electrophoresis, including, without limitation, capillary electrophoresis, free-flow electrophoresis, non-denaturing (native) gel electrophoresis and denaturing gel electrophoresis, mass spectrometry, and chromatofocusing. In these methods, the native protein standards are either added to a sample that comprises a protein (preferably in nondenatured form), or analyzed or separated side-by-side or in sequence with the sample using the separation or analysis methods. The intensity of signal or separation time or distance of one or more native protein standards can be compared with the same parameters of one or more sample proteins to obtain a relative or calculated value for the mass, size, concentration, or electrical charge or charge to mass ratio of one or more sample proteins.

Exemplary Liquid Native Protein Molecular Weight Marker Sets

The inventions provide proteins for use in native protein molecular weight marker sets that are 1) stable for at least one month at 4 degrees Centigrade; 2) stable for at least one year at −20 degrees Centigrade; and 3) exhibit sharp (non-diffuse) band resolution when electrophoresed on Tris-glycine and Blue Native gel systems. The identified proteins include: IgM isolated from bovine serum; apoferritin isolated from equine spleen; B-phycoerythrin; lactate dehydrogenase isolated from porcine heart; bovine serum albumin; and soybean trypsin inhibitor. The molecular weights of these proteins in native form are provided in Table 1.

The proteins used as markers can be obtained from any of various sources, for example, Immunoglobulin M, Bovine Plasma, from Sigma I8135; Immunoglobulin M, Bovine Plasma, from Accurate Chemical AIM07; Immunoglobulin M, Mouse, from Invitrogen (Zymed) 02-6800; Immunoglobulin M, Rat, from Invitrogen (Zymed) 02-9888; Apoferritin, Equine Spleen, from Sigma A3660; Apoferritin, Equine Spleen, from Calbiochem 178440; Apoferritin, Equine Spleen, from MP Bio 100260; B-Phycoerythrin, Unicellular Red Alga, from Alexis 610-145-M002; B-Phycoerythrin, Porphyridium sordidum or Porphyridium cruendum, from Cyanotech 100301; Lactate Dehydrogenase, Porcine Heart, from Calbiochem 427211; Lactate Dehydrogenase, Bovine Heart, from Sigma L3916; Lactate Dehydrogenase, Porcine Heart, from Sigma L7525; Lactate Dehydrogenase, Rabbit Muscle, from Calbiochem 427217; Albumin, Bovine Serum, from Sigma A2153; Albumin, Bovine Serum, from Sigma A0281; Albumin, Bovine Serum, from Sigma A3059; Albumin, Bovine Serum, from Sigma A8531; Albumin, Bovine Serum, from Fisher BP1605-100; Albumin, Bovine Serum, from VWR EM-2905; Albumin, Bovine Serum, from Boehringer Mannheim 238040; Trypsin Inhibitor, Soybean, from Fluka (Sigma) 93619; Trypsin Inhibitor, Soybean, from Fluka (Sigma) 93620; Trypsin Inhibitor, Soybean, from USB 21730; Trypsin Inhibitor, Soybean, from Sigma T9003; Trypsin Inhibitor, Soybean, from Calbiochem 650357; and Trypsin Inhibitor, Soybean, from Amresco K213.

TABLE 1
Protein Components of a Liquid Native Marker Set

| | | Quaternary | Subunit Sizes | Native Size |
|---|---|---|---|---|
| Protein | Source | Structure | (kDa) | (kDa) |
| Immunoglobulin M | Bovine Plasma | Hexamer (μ₂λ₂)₆ | μ = 76, λ = 27, | Hexamer 1236 |
| Saini et al. (1999), | | Pentamer (μ₂λ₂)₅j | j = 18 | Pentamer 1048 |
| Wiersma et al. (1998), | | | | |
| Accession #NP_786967 | | | | |
| Apoferritin | Equine Spleen | α_{24,} α₂₄ + α₁₂ | α = 20 | 720 (minor band) |
| Accession # P02791 | | | | 480 (major band) |
| B-Phycoerythrin | Porphyridium | (αβ)₆γ | α = 16.7, β = 18.6, | 242 |
| Glazer and Hixson | sordidum | | γ = 30 | |
| (1977), Accession # | | | | |
| S27327 and S27326 | | | | |
| Lactate | Porcine heart | α₄ | α = 36.6 | 146 |
| Dehydrogenase | | | | |
| Accession #P00336 | | | | |
| Albumin | Bovine Serum | Single subunit | α = 66.4 | 66 |
| Accession #P02769 | | | | |
| Trypsin Inhibitor | Soybean | Single subunit | α = 20.5 | 20 |
| Accession #CAA56343 | | | | |

Two or more of the listed proteins can also be used in any combination as a liquid native marker set. A preferred formulation of a liquid native protein marker set includes bovine plasma IgM at 1 mg/mL, equine spleen apoferritin at 0.4 mg/mL, algal B-phycoerythrin at 0.2 mg/mL, porcine heart lactate dehydrogenase at 0.2 mg/mL, bovine serum albumin at 0.2 mg/mL, and soybean trypsin inhibitor at 0.2 mg/mL.

Any of these proteins, selected for their stability, shelf-life, and property of giving nondiffuse bands on native polyacrylamide gels, can also be used independently to compare the migration in a native gel of the marker protein with that of one or more sample proteins. The present invention includes methods of determining the relative native molecular weight of one or more proteins using bovine serum IgM as a molecular weight marker on the same native gel for comparison. The present invention also includes methods of determining the relative native molecular weight of one or more proteins using equine spleen apoferritin as a molecular weight marker on the same native gel for comparison. The present invention also includes methods of determining the relative native molecular weight of one or more proteins using B-phycoerythrin as a molecular weight marker on the same native gel for comparison. The present invention also includes methods of determining the relative native molecular weight of one or more proteins using porcine heart lactate dehydrogenase as a molecular weight marker on the same native gel for comparison. The present invention also includes methods of determining the relative native molecular weight of one or more proteins using bovine serum albumin as a molecular weight marker on the same native gel for comparison. The present invention also includes methods of determining the relative native molecular weight of one or more proteins using soybean trypsin inhibitor as a molecular weight marker on the same native gel for comparison.

Kits with Liquid Native Markers

Liquid native marker sets can be sold in kits, where the kits include at least one tube or vial containing native protein molecular weight markers as a liquid mixture of two or more proteins. A tube or vial can contain between 5 ul and 5 mL of liquid native markers, such as between 10 ul and 2.5 mL, such as between 25 ul and 1 mL, or between 100 uL and 500 ul.

In preferred embodiments, a liquid native protein marker kit includes marker proteins in a nondenaturing buffer compatible with native electrophoresis (for example, Tris-glycine native gels, Tris-actate native gels, or Blue Native gels), in which a nondenaturing “heavy” compound (such as glycerol) and, optionally, a dye can be present for convenient loading into a sample well. Other compounds, such as but not limited to salts, antioxidants, antimicrobial agents (for example, sodium azide), reducing agents, chelating agents, or protease inhibitors can also optionally be present. The liquid marker protein solution provided in a kit can also include one or more nondenaturing detergents. An exemplary solution for a liquid native marker set includes 50 mM BisTris/HCl pH 7.0, 50mM NaCl, 10% w/v Glycerol, 0.001% Ponceau S, and sodium azide.

The invention provides in certain embodiments a kit comprising at least one container containing a liquid native protein molecular weight marker set comprising a solution of at least two proteins of different molecular weights and in their native conformation, wherein the migration of the at least two proteins on nondenaturing gels is a function of their molecular weights, and wherein the liquid native protein molecular weight marker set is stable for at least one month at 4 degrees Centigrade, and a second container containing the liquid native protein molecular weight marker set and/or at least one native gel reagent, wherein the at least one native gel reagent is Coomassie G-250 dye, a non-denaturing sample buffer, a nondenaturing detergent, or a pre-cast non-denaturing gel.

Preferably, the liquid native marker set of the kit is provided at a concentration such that an aliquot of the protein marker set can be loaded directly on the gel without further dilution, although for some applications markers can be diluted before electrophoresis. For example, each of said two or proteins of said liquid native protein molecular weight marker set can be present at a concentration of from 0.05 mg/ml to 5 mg/mL, or from 0.1 mg/ml to 2 mg/mL.

The liquid protein markers can be in a formulation such that between 0.5 ul and 50 ul can be loaded in a single lane of a gel and visualized by staining with a Coomassie, SYPRO, or silver stain, and preferably between 1 ul and 25 ul can be loaded on a single lane of a gel and visualized by staining with a Coomassie, SYPRO, or silver stain.

The liquid marker set can be provided in a kit that comprises two or more containers. In this way, the user can store one of the containers at 4 degrees Centigrade and additional containers can be stored at −20 degrees C. for longer term storage. For example, a kit can provide two, three, four, five, or more containers, so that the user can conveniently keep a first container at 4 degrees C. for immediate use that does not require thawing the markers, and store the remainder at −20 degrees C. until the first container is depleted.

Marker sets having different protein compositions can be included in a single kit. For example, different marker sets can include a set of proteins that have a different molecular weight range.

The kits can further include at least one protein purification, isolation, or preparation reagent or at least one gel reagent, such as, for example, a sample or protein solubilizing buffer, a nondenaturing detergent (for example, dodecylmaltoside, octylglucoside, digitonin), Coomassie® G-250, gel loading buffer, an electrophoresis running buffer, a pre-cast native gel, or a gel stain. The pre-cast native gel can be, for example, a Tris-glycine gel, a Tris-acetate gel, or a Bis-tris gel. The kit can also include an instruction sheet that contains information on a) the use of the markers in electrophoresis and, preferably, b) the use of the markers in estimating the molecular weight of one or more sample proteins electrophoresed on the same gel as the liquid native marker set. Alternatively, the instruction sheet can refer the user to a web site that provides instructions for a), b), or both.

Methods of Generating Revenue

In certain aspects of the invention, a liquid native protein marker set is provided that is a commercial product that is sold using a stream of commerce. The commercial product is typically sold with a label and/or in a kit. The liquid native protein marker set is offered for sale by a provider, such as a for-profit business entity, to a customer. The commercial product in preferred aspects is a ready-to-use commercial formulation, in which at least two proteins of a native protein standard set in liquid solution are provided by a commercial supplier to a user in frozen or non-frozen liquid form. The commercial molecular weight marker set can be provided in an appropriate buffer and at a concentration and such that at least a portion of the liquid protein molecular weight marker set can be loaded directly on a gel.

In another embodiment, provided herein is a method of generating revenue by selling a native protein molecular weight marker set in a liquid form. The method includes providing a means to purchase a liquid native protein molecular weight marker set for native electrophoresis, wherein the liquid native protein molecular weight marker set includes a solution of at least two proteins of different molecular weights in their native conformation. The method can further include activating the means to purchase the liquid native protein molecular weight marker set and entering payment information. Furthermore, the method can include payment from a customer to a provider of the marker set. The set can include, 2, 3, 4, 5, 6, 7, 8, 9, 10, 11, 12, 13, 14, 15, 20 or 25 proteins.

The means to purchase the marker set is a purchasing function that can include any of the means in methods used by biological research reagent companies to sell molecular weight markers. The method can include a telephonic system and/or an computer-based system. As a non-limiting example, the method can include displaying a link to purchase the molecular weight marker set on an Internet page or other displayed page on a local or wide area network. In addition, or alternatively, the means can be a telephone or text message ordering system. Another means, can include a direct order placed via traditional mail or an order placed verbally in person, for example with a salesperson. The liquid native markers can be stocked in a supply center, in which a customer can remove one or more containers containing liquid native markers and record the amount of product taken on a page, in a book or ledger, or using a computer that is part of the stock center or accessed via the customer's personal computer (PC). The removal of product and recording of the removal of product can be performed by the purchaser or by an employee stock center or supplier of the product. The recording of the removal of the product constitutes an agreement on the part of the customer to pay for the liquid native markers. Regardless of the means, typically the customer uses the means to purchase the molecular weight marker set.

To purchase the marker set the customer gives consideration to the provider. Money is usually the form of consideration for the purchase paid by the customer to the provider. In exchange for the consideration, the provider who is typically an outside vendor, ships the marker set to the customer, typically an end-user customer. In the case of a stock center, an outside vendor ships to a stock center, typically within a research institution or company, and the purchaser removes the liquid native marker set and subsequently pays for the purchase, typically after receiving a bill generated by the supplier from the product removal record. It will be understood that the customer can be any customer that typically purchases native protein gels. For example, the customer can be a researcher at a research entity such as a research institute or a commercial entity. The customer can also be a medical diagnostics or pharmaceutical company.

The marker set can include, for example, between two and fifteen proteins, and in certain illustrative examples includes between six and ten proteins having molecular weights between 5 kDa and 2000 kDa, or between 8 and 1500 kDa. Each of the proteins typically has a different molecular weight that can be distinguished on a native polyacrylamide gel.

The markers can cost, for example, between $0.5 and $20 per Coomassie G250 stained gel lane, preferably between $1 and $10. For example, a tube of markers can include 5 microliters (ul) to 2500 ul of the liquid marker set, which can cost between $1 and $5000, usually between $50 and $2500. For example a tube can include 200 ul to 300 ul of a ready to use liquid marker set at a concentration such that 2-10 ul are loaded onto a gel lane to be visualized by Coomassie blue staining. The cost for the 200 ul to 300 ul tube can be, for example, $50 to $500 U.S. dollars.

The purchasing function can be used to purchase additional products that are directly or indirectly related to the molecular markers provided herein. For example, the purchasing function can further be used to purchase a non-denaturing gel, such as a pre-cast gel of a single or gradient acrylamide concentration of between 3 and 20% acrylamide (including, without limitation, native Tris-glycine gels (e.g., Novex® Tris-Glycine gels (Invitrogen, Carlsbad, Calif.)), native Tris-acetate gels (e.g., NuPAGE® Novex® Tris-Acetate gels (Invitrogen, Carlsbad, Calif.)), and Bis-tris gels (e.g., NativePAGE™ Novex® Bis-Tris gels (Invitrogen, Carlsbad, Calif.)); and/or stains, such as a Coomassie® blue stain, such as but not limited to Coomassie® G-250. Other stains that can be included in a purchase that includes the purchase of liquid native markers are, without limitation, copper protein gel stains, silver protein gel stains, and SYPRO® gel stains. The related product can also be an electrophoresis running buffer.

The liquid native protein molecular weight marker sets sold to a customer are discussed herein. For example, the liquid native protein molecular weight marker set is typically stable for at least one month at 4 degrees Centigrade, and in certain aspects is stable for at least 2, 3, 4, 5, 6, 7, 8, 9, 10, 11, or 12 months at 4 degrees Centigrade or −20 degrees Centigrade up to between 2, 3, 4, 5, 6, 7, 8, 9, 10, 11, 12, 13, 14, 15, 16, 17, 18, 24, 36, 48, and 60 months or longer at 4 degrees Centigrade or −20 degrees Centigrade.

The method can further include shipping the liquid native protein molecular weight marker set. For example, the shipping can include shipping using interstate commerce. The shipping is typically done by a provider to a customer. The customer is typically not in the same building as the provider. The shipping typically performed by a commercial carrier or a governmental entity, such as the U.S. Postal Service.

Another embodiment provided herein is a method for generating revenue, comprising: providing a customer with a purchase function to purchase a liquid native protein molecular weight marker set comprising a solution of at least two proteins, wherein when the purchase function is used to purchase the liquid native protein molecular weight marker set, revenue is generated by a provider of either or both the purchase function and the liquid native protein molecular weight marker set.

In another embodiment, the present invention also provides a method for selling a liquid native protein molecular weight marker set and/or kit provided herein, comprising: presenting to a customer an input function of a telephonic ordering system, and/or presenting to a customer a data entry field or selectable list of entries as part of a computer system, wherein the liquid native protein molecular weight marker set and/or kit is identified using the input function. Where the input function is part of a computer system, such as displayed on one or more pages of an Internet site, the customer is typically presented with an on-line purchasing function, such as an online shopping cart, wherein the purchasing function is used by the customer to purchase the identified liquid native protein molecular weight marker set, and/or kit. In one aspect, a plurality of identifiers are provided to a customer, each identifying a liquid native protein molecular weight marker set, and/or a kit provided herein in different volumes, or along with a related product such as a precast native gel or a dye, such as a Coomassie® stain. The method may further comprise activating the purchasing function to purchase the liquid native protein molecular weight marker set and/or kit provided herein.

In illustrative examples, methods for generating revenue include offering to sell, such as by providing a link to a purchase function, and/or selling a liquid native protein marker set that includes two or more proteins in a ready-to-use formulation. Therefore, in illustrative examples according to the methods for generating revenue provided herein, the native protein marker set is in a liquid buffer compatible with native electrophoresis (for example, Tris-glycine native gels, Tris-acetate native gels or Blue Native gels), in which a nondenaturing “heavy” compound (such as glycerol) and, optionally, a dye is present for convenient loading into a sample well. Other compounds, such as but not limited to salts, antioxidants, antimicrobial agents, reducing agents, chelating agents, or protease inhibitors can also optionally be present. The liquid marker protein solution can also include one or more nondenaturing detergents. Preferably, the buffer in which the proteins are dissolved is at or near neutrality, that is at a pH greater than 6 and less than 8, and more preferably at a pH between about 6.5 and 7.5. An exemplary solution for a liquid native marker set is 50 mM BisTris/HCl pH 7.0, 50 mM NaCl, 10% w/v Glycerol, 0.001% Ponceau S. The solution can optionally include an antimicrobial agent, such as for example sodium azide. The purchase function can be a telephonic purchase function or a purchase function provided on an Internet page, such as part of an Internet shopping cart function.

The present invention also includes a method of generating revenue by selling native standards. The method includes providing a customer with a liquid protein molecular weight marker set of the invention; providing the customer with either an instruction sheet providing the molecular weights of said two or more proteins of said liquid protein molecular weight marker set or a world wide web address of web site available by internet access, where the website provides molecular weights of said two or more proteins of said liquid protein molecular weight markers set, or both: and receiving revenue from the customer in exchange for providing the liquid protein molecular weight markers set. In preferred embodiments, the instruction sheet, the website, or both, provide instructions for calculating the molecular weights of proteins using the liquid native molecular weight markers as electrophoresis standards, and/or provide images of exemplary gels showing gel bands of the molecular weight markers.

Preferably, the liquid native markers are ordered and provided to the customer as a kit. The method of generating revenue can also include providing the customer with a web site through which the customer can order the liquid native molecular weight marker set. The web site also can electronically record the transaction and generate a sales bill.

The following examples are intended to illustrate but not limit the invention.

### EXAMPLE I

### Electrophoresis of a Liquid Native Marker Set

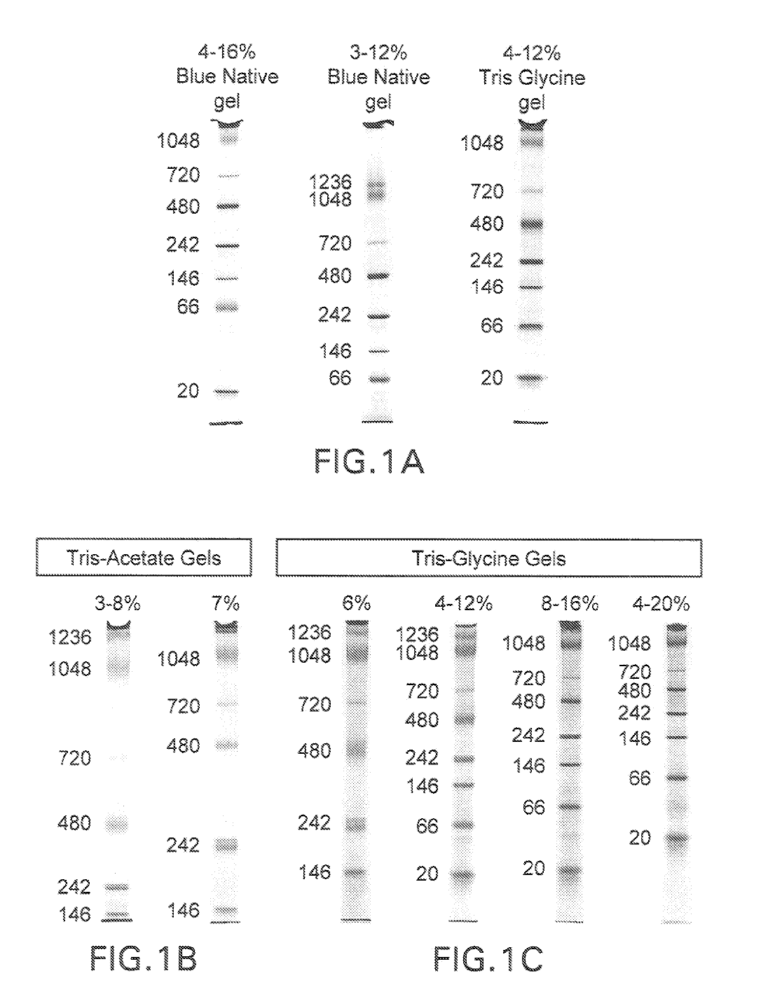

A liquid native protein marker set was made that contained the proteins depicted in Table 1 at the following concentrations: 1 mg/mL IgM, 0.4 mg/mL apoferritin, 0.32 mg/mL B-phycoerythrin, 0.3 mg/mL lactate dehydrogenase, 0.2 mg/mL BSA, and 0.5 mg/mL soybean trypsin inhibitor. The liquid native marker (LNM) set was electrophoresed on three different gel systems. In each case, 5 microliters of the marker formulation (in 50 mM BisTris/HCl pH 7.0, 50 mM NaCl, 10% w/v Glycerol, 0.001% Ponceau S) were loaded directly on the gel. FIG. 1A shows the liquid native marker set electrophoresed on different gel systems and stained with Coomassie® G-250 dye (Colloidal Blue Staining Kit, Invitrogen, Carlsbad, Calif.). In the leftmost lane, the marker set was run on a 4-16% Blue Native acrylamide gel. In the middle lane the marker set was run on a 3-12% Blue Native acrylamide gel. The Blue Native gels were run at 150V constant for 90 minutes with running buffer of 50 mM BisTris, 50 mM Tricine. In the rightmost lane the markers were run on a 4-12% Tris glycine gel with an operating pH of approximately 9.3. The Tris Glycine gel was run at 125V constant for 111 minutes with running buffer of 25 mM Tris, 192 mM Glycine.

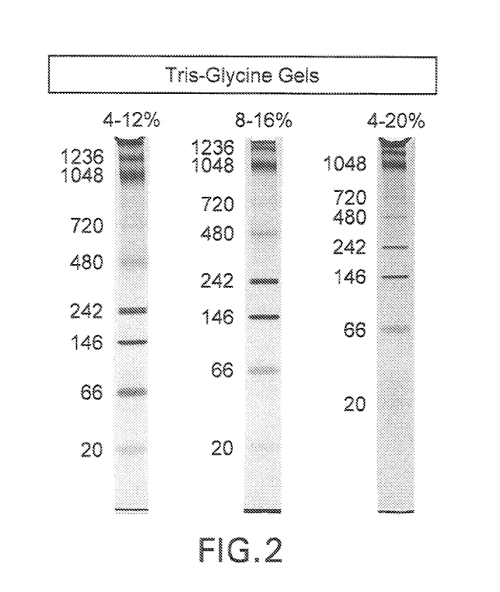

FIG. 1B shows the same marker formulation electrophoresed on 3-8% and 7% acrylamide Tris Acetate gels, and FIG. 1B shows the same marker formulation electrophoresed on 4-12%, 8-16%, and 4-20% acrylamide Tris-Glycine gels. In both cases, the gels were stained with Coomassie® G-250 dye (Colloidal Blue Staining Kit, Invitrogen, Carlsbad, Calif.). FIG. 2 shows the same liquid native marker (LNM) formulation, diluted to 0.05× of its stock concentration and electrophoresed on 4-12%, 8-16%, and 4-20% acrylamide Tris-Glycine gels and silver stained (SilverQuest™ protein gel stain).

The figures show that the markers provide sharp bands that migrate in order of their molecular weights without producing substantially detectable non-marker bands in several gel systems.

### EXAMPLE II

### Migration of a Liquid Native Market Set in Different Gel System

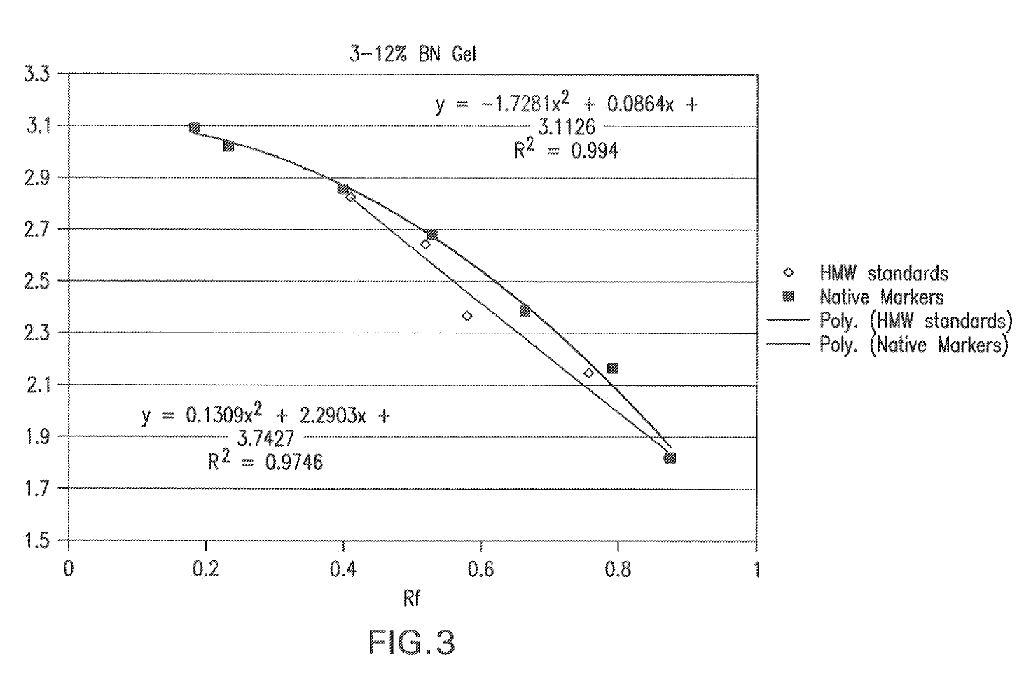

A liquid native protein marker (LNM) set formulated as in Example 1 was run on a 3-12% blue native (BN) PAGE gel alongside a commercially available marker set (“HMW marker” which is provided to the customer in lyophilized form). The plot of log MW vs. Rf for proteins from HMW marker and the liquid native marker unstained native protein standard from the gel is shown in FIG. 3. The standard curve lines were plotted using a second-order polynomial best fit; the equation for the HMW marker curve was y=1.7281x²+0.864x+3.1126; the equation for the LNM set was y=0.1309x²−2.2903x+3.7427. The R-squared value for the commercially available marker set was 0.9746. The R-squared value for the LNM set of Example 1 was 0.994.

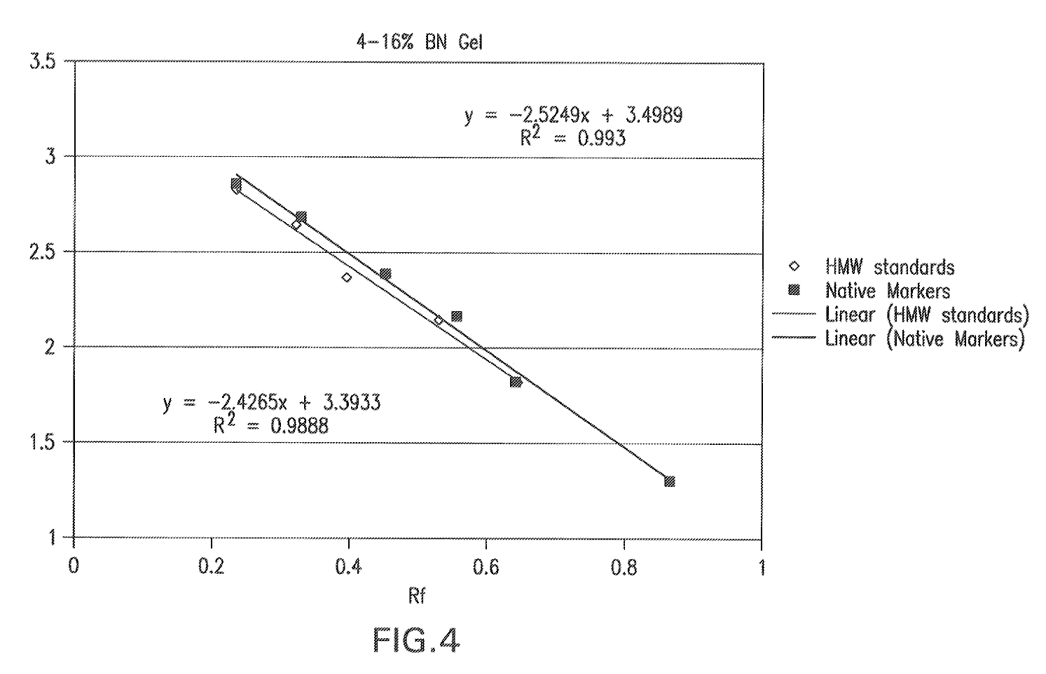

FIG. 4 shows the same two marker sets electrophoresed on a 4-16% Blue Native gel. The standard curve lines were plotted using a linear best fit; the equation for the HMW marker curve was y=−2.4265x+3.3933; the equation for the LNM set was y=−2.5249x+3.4989. The R-squared value for the commercially available “HMW marker” set was 0.9888. The R-squared value for the liquid native marker set of Example 1 was 0.993.

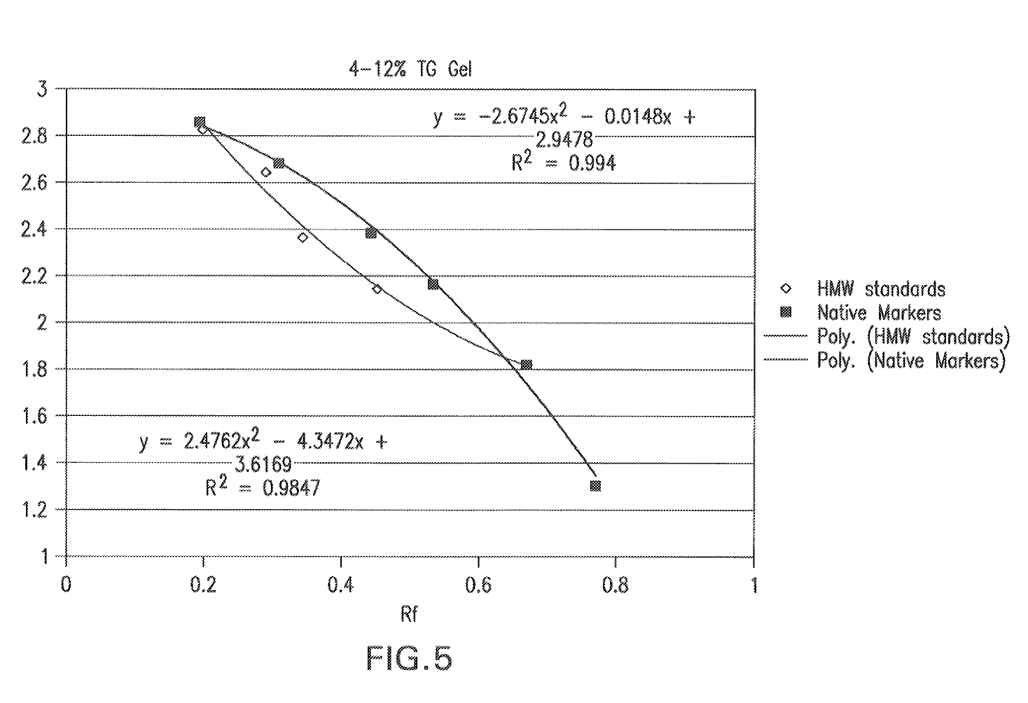

FIG. 5 shows the same two marker sets electrophoresed on a 4-12% Tris-Glycine gel. The standard curve lines were plotted using a linear best fit; the equation for the HMW marker curve was y=2.4762x²−4.3472x+3.6169; the equation for the LNM set was y=−2.6745x²−0.0148x+2.9478. The R-squared value for the commercially available “HMW marker” set was 0.9847. The R-squared value for the liquid native marker set of Example 1 was 0.994.

### EXAMPLE III

### Stability of a Liquid Native Market Set

A new lot of native liquid markers of the invention as described in Example 1 was run next to a previously passed lot of LNM having the composition provided in Example 1, 5 uL per lane, on both 3-12% and 4-16% Blue Native gels. After staining with the Colloidal Blue Staining kit, the gels were scanned as 300 dpi .tiff images and saved as both color and grayscale images. Image analysis (densitometry) is performed by TotalLab software to provide migration (Rf) and peak height values for all bands detected in sample lanes. QC specifications for the LNM were defined as:
Migration=Rf for marker band in new lot must match Rf for marker band in previously passed lot loaded in an adjacent lane with a tolerance of +/−0.02 Rf units
Minor bands=The peak height for any non-marker band may not be greater than 20% of the smallest of the two nearest marker band peak heights.
Visual=Visual inspection of the new lot does not reveal any other differences in performance when compared to the previously passed lot.

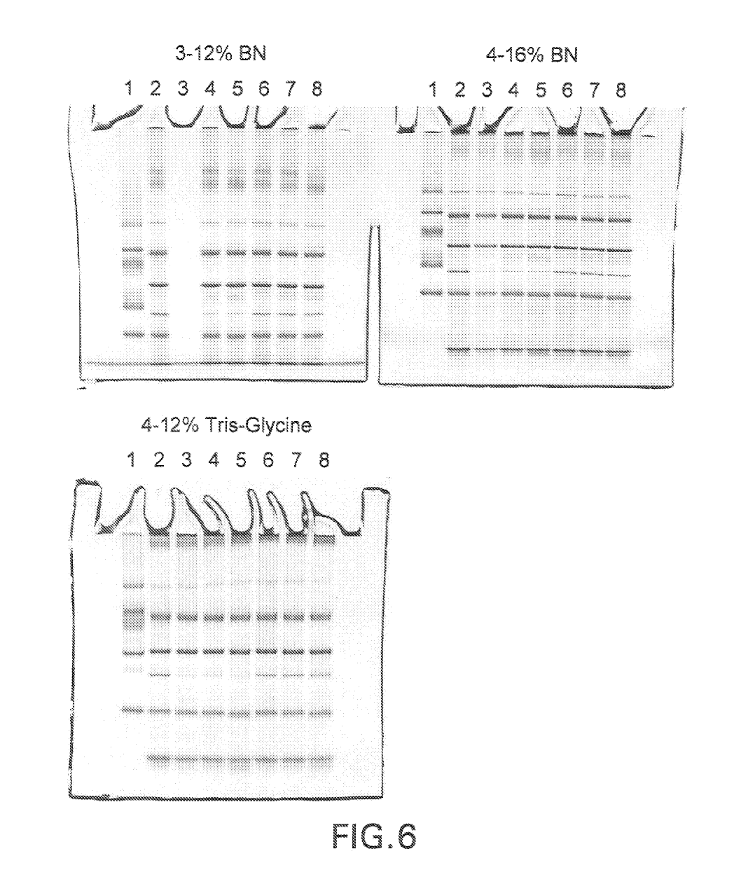

Aliquots of different lots of LNM were stored at different temperatures to simulate accelerated aging and then 5 microliter aliquots were run on Blue Native and Tris Glycine gels (FIG. 6). The stained gel images were analyzed by densitometry with TotalLab software to determine pass or fail status according to QC criteria as stated in methods. Lane 1 contained commercially available molecular weight markers; Lane 2, LNM lot A, stored at 4 degrees C. for 91 days; Lane 3, LNM lot B, stored at 4 degrees C. for 67 days; Lane 4, LNM lot B, stored at 22 degrees C. for 67 days; Lane 5, LNM lot B, stored at 30 degrees C. for 67 days; Lane 6, LNM lot C, stored at 4 degrees C. for 66 days; Lane 7, LNM lot C, stored at 22 degrees C. for 66 days; and Lane 8, LNM lot C, stored at 30 degrees C. for 66 days.

The LNM lot C passed criteria for stability on all gels at the 22° C. for 66 days timepoint (FIG. 6, lane 7) but did not meet criteria at the 30° C. for 66 days timepoint (FIG. 6, lane 8). The LNM lot B (FIG. 6, lanes 3-5) did not meet criteria due to improper sample load of the LDH band. Problematic bands that appear upon extended storage result in the failure to meet criteria when their peak heights become greater than 20% of a neighboring marker band's peak height. While the problematic bands are visible at the 22° C. for 66 days timepoint for Lot C (FIG. 6, lane 7), they are still faint enough that they can be considered minor and therefore stability requirements are met. According to the equation stated in methods for accelerated aging, 22° C. for 66 days is equivalent to storage at −20° C. for 1213 days (3.3 years) or 4° C. for 230 days (7.6 months). The markers thus have a stability of at least three years when stored at −20° C., or at least six months when stored at 4° C.

All references cited herein are incorporated by reference in their entireties. Headings used herein are for convenience only and are not intended to limit the invention. Disclosure within a section with a heading can be applicable to disclosure within another section with a different heading. Although the invention has been described with reference to the above examples, it will be understood that modifications and variations are encompassed within the spirit and scope of the invention.

1. A liquid native protein molecular weight marker set for native electrophoresis, comprising a solution of at least two proteins of different molecular weights and having a native conformation, wherein the migration of the proteins of the marker set on nondenaturing gels is a function of their molecular weights, and wherein proteins of the liquid native protein molecular weight marker set are stable for at least one month at °20 degrees Centigrade.

2. The liquid native protein molecular weight marker set of claim 1, wherein said liquid native protein molecular weight marker set is a commercial product.

3. The liquid native protein molecular weight marker set of claim 2, wherein said liquid native protein molecular weight marker set is a ready-to-use commercial formulation.

4. The liquid native protein molecular weight marker set of claim 1, wherein said at least two proteins are stable in the solution for at least one month at 4 degrees Centigrade.

5. The liquid native protein molecular weight marker set of claim 1, wherein said at least two proteins are stable in the solution for at least one year at −20 degrees Centigrade.

6. The liquid native protein molecular weight marker set of claim 1, wherein at least one of said at least two proteins is a protease inhibitor.

7. The liquid native protein molecular weight marker set of claim 6, wherein said protease inhibitor is a trypsin inhibitor.

8. The liquid native protein molecular weight marker set of claim 7, wherein said protease inhibitor is soybean trypsin inhibitor.

9. The liquid native protein molecular weight marker set of claim 1, wherein at least one of said at least two proteins comprises at least one chromophore or at least one fluorophore.

10. The liquid native protein molecular weight marker set of claim 9, wherein said at least one of said at least two proteins comprises a chromophore.

11. The liquid native protein molecular weight marker set of claim 10, wherein said at least one protein is phycocyanin, allophycocyanin, or phycoerythrin.

12. The liquid native protein molecular weight marker set of claim 11, wherein said at least one protein is B-phycoerythrin.

13. The liquid native protein molecular weight marker set of claim 1, wherein at least one of said at least two proteins has a native molecular weight of greater than 700 kDa.

14-15. (canceled)

16. The liquid native protein molecular weight marker set of claim 1, wherein at least one of said at least two proteins has a molecular weight of at least one megadalton.

17-21. (canceled)

22. The liquid native protein molecular weight marker set of claim 1, comprising at least three proteins of different molecular weights.

23. The liquid native protein molecular weight marker set of claim 22, comprising at least four proteins of different molecular weights.

24. The liquid native protein molecular weight marker set of claim 23, comprising at least five proteins of different molecular weights.

25. The liquid native protein molecular weight marker set of claim 24, comprising at least six proteins of different molecular weights.

26. The liquid native protein molecular weight marker set of claim 25, comprising IgM, apoferritin, B-phycoerythrin, lactate deyhdrogenase, BSA, and soybean trypsin inhibitor.

27. The liquid native protein molecular weight marker set of claim 1, wherein the liquid native protein molecular weight marker set has a shelf-lief of at least one month at 4 degress Centrigrade.

28-41. (canceled)

42. A method of determining the estimated or relative molecular weight of at least one protein under native conditions, comprising:
applying to a native gel, a sample comprising a protein and a sample comprising a liquid protein molecular weight marker set comprising a solution of at least two proteins, wherein the proteins of the liquid native protein molecular weight marker set are stable for at least one month 4 degrees Centigrade;
electrophoresing the sample and the liquid protein molecular weight marker set in the native gel; and
comparing the migration of one or more proteins in the sample to one or more proteins of the liquid protein molecular weight marker set, thereby determining the estimated or relative molecular weight of at least one protein under native conditions.

43-58. (canceled)

59. A method of generating revenue by selling native protein molecular weight standards, comprising:
a) providing a customer with the liquid native protein molecular weight marker set for native electrophoresis, wherein the liquid native protein molecular weight marker set comprises a solution of at least two proteins of different molecular weights and in their native conformation.

60. The method of claim 59, wherein the liquid native protein molecular weight marker set is stable for at least one month at 4 degrees Centigrade.

61-90. (canceled)
